(19) United States
(12) Patent Application Publication
(10) Pub. No.: US 2009/0253786 A1
(43) Pub. Date: Oct. 8, 2009

(54) Synthesis, anti-allergic, anti-asthmatic and anti-inflammatory activities of CEE-1 and CEE-2

(75) Inventors: Charles I. Ezeamuzie, Kuwait City (KW); Ivan Edafiogho, Kuwait City (KW)

Correspondence Address: FURR LAW FIRM, 2622 DEBOLT ROAD, UTICA, OH, 43080, US

(73) Assignee: UNIVERSITY OF KUWAIT, Safat (KW)

(21) Appl. No.: 12/098,460

(22) Filed: Apr. 7, 2008

(51) Int. Cl. A61K 31/24; A61P 11/06; A61P 37/08; A61P 19/02

(52) U.S. Cl. 514/535

(57) ABSTRACT

“Enhydrazone esters of formula (I): wherein R is methyl or ethyl group have been synthesized. The compounds of formula (I) are potent inhibitors of degranulation of both mast cells and eosinophils. These compounds also suppress cytokines generation by leukocytes. These biological activities are relevant to the prevention and/or treatment of asthma, allergies and inflammatory diseases. It is known to the art that inhibitors of mast cell degranulation such as cromolyn sodium and nedocromil sodium are used in the treatment of asthma and other allergic diseases. Inhibitors of cytokine generation, such as steroids, are also useful in the prevention and treatment of inflammatory diseases as well as asthma and allergies”.

### FIELD OF THE INVENTION

The field of the present invention relates generally to discovery of two new enhydrazone esters (CEE-1 and CEE-2) with potential for the treatment of allergic diseases, asthma and inflammation.

### BACKGROUND OF THE INVENTION

Allergic diseases, such as asthma, allergic rhinitis, urticaria, atopic eczema/dermatitis affect up to 25% of mankind, and the prevalence appears to be increasing world-wide. [Sly, 1999; Masoli et al., 2004]. Eosinophils and mast cells are the two most important pro-inflammatory cells involved in the pathophysiology of asthma and other allergic diseases. The degranulation of these cells and the release of many mediators, including the tissue-damaging proteins by eosinophils, orchestrate the inflammation that underlies these diseases.

The primary trigger of allergic diseases is the interaction between an allergen and the specific immunoglobulin E (IgE) molecules bound to the high affinity IgE receptor (FcεRI) on the surface of mast cells of sensitized patients. This results in the degranulation of these cells and the consequent release of pre-formed alLergic and inflammatory mediators such as histamine, tryptase and various enzymes [Prussin & Metcalfe 2006]. In addition, other mediators such as eicosanoids (eg, leukotrienes) and. cytokines (especially TNF-α, IL-4, IL-13 and GM-CSF) are synthesized and released on activation [Schwartz et al., 2002]. All of these mediators participate in the pathophysiology and chronicity of the diseases.

Like the mast cells, eosinophils are also intimately involved in the pathophysiology of asthma [Rothenberg and Hogan 2006]. Histologically, the asthmatic lung is heavily infiltrated by eosinophils which are believed to degranulate at the site to release mediators that contribute to bronchial inflammation, airway hyperresponsiveness and airway tissue remodeling. Eosinophil granules contain several cationic proteins such as major basic protein (MBP), eosinophil cationic protein (ECP), eosinophil-derived neurotoxin (EDN) and eosinophil peroxidase (EPO) which are directly toxic to many tissues, [Hogan 2007]. The released cationic proteins are believed to damage, in particular, the epithelium leading to bronchial hyperresponsiveness that is so characteristic of asthma. [Fravehan et al., 1988; Rothenberg & Hogan 2006]. In fact, sputum eosinophil count as well as the level of eosinophil cationic protein have been shown to be a reliable indicator of disease severity or exacerbation [Green et al., 2002]. Furthermore, results from studies employing eosinophil-deficient mice strongly supported an important role for eosinophils in asthma [Fulkerson et al., 2006].

In view of the direct roles that mast cells and eosinophils play in the pathophysiology of asthma, these cells have been the target of the search for better anti-asthma drugs, especially the inflammatory aspect. It is currently believed that an ideal anti-asthma drug should be able to suppress the activities of both mast cell and eosinophils, especially their degranulation. In a previous study [Ezeamuzie and Al-Hage, 1998], we evaluated the effect of 8 clinically used anti-asthma drugs, including the steroid dexamethasone but, amazingly, none was found to directly inhibit acute eosinophil degranulation at therapeutically relevant concentrations. Thus, the development of anti-asthma and anti-allergic drugs capable of blocking the degranulation of both mast cells and eosinophils has continued to be a research priority.

It is well known to the art that inhibitors of mast cell degranulation, such as cromolyn sodium and nedocromil sodium are used in the treatment of asthma and other allergic diseases [Parnham 1996; Konig et al., 2000; Leino et al., 1992]. Also drugs that may inhibit eosinophil accumulation or inhibit cytokine generation, such as steroids, are also anti-asthma and anti-allergic drugs [Weltman 1999; Lumry 19999]. However, no currently available drug has the ability to directly inhibit the degranulation of both mast cells and eosinophils at clinically relevant concentrations.

Even steroids, which are the current mainstay of anti-inflammatory therapy, lack an acute and direct effect on degranulation of these cells. Steroids also have many side effects.

For an optimal anti-inflammatory treatment of asthma and other allergic diseases, an ideal drug should inhibit the degranulation of these two cell types as well as prevent the release of cytokines and other inflammatory mediators from pro-inflammatory cells such as macrophages.

### SUMMARY OF THE INVENTION

In view of aforementioned problems we hereby propose to provide a solution using the principles of drug design to synthesize two new compounds CEE-1 and CEE-2 which have activities relevant to the treatment of asthma, allergy and inflammation.

CEE-1 and CEE-2 are enhydrazone esters which uniquely combine three important attributes: ability to act directly and potently to inhibit human eosinophil degranulation, ability to inhibit directly and potently the degranulation of allergen-stimulated degranulation of mast cells and the ability to strongly and directly inhibit the release of tumor necrosis factor-alpha (TNF-α) in lipopolysacchrides (LPS)-stimulated macrophages.

These two novel compounds have a unique combination of properties that make them potentially useful in the treatment of asthma and other allergic diseases. They are also potentially useful in other non-allergic inflammatory diseases, such as rheumatoid arthritis.

### BRIEF DESCRIPTION OF THE DRAWINGS

The above and other aspects, features, and advantages of the present invention will be better and more fully understood by those skilled in the art with reference to the following detailed an more particular description of specific and preferred embodiments thereof, resented in conjunction with the following drawings to show how the same may be carried into effect, wherein:

FIG. 1 displays the Inhibition of complement C5a-stimulated degranulation (EPO release) of human eosinophils by CEE-1 and CEE-2;

FIG. 2 displays the Inhibition of antigen induced IgE-mediated degranulation (β-hexoseaminidase release) of mast cells (RBL-2H3) by CEE-1 and CEE-2. Cells were incubated with the drugs for 10 min before stimulation;

FIG. 3 displays the Inhibitory effect of CEE-1 and CEE-2 on the release of TNF-α from LPS-stimulated mouse peritoneal macrophages; and

FIG. 4 displays a scheme of the synthesis of compounds CEE-1 and CEE-2.

### DETAILED DESCRIPTION OF THE INVENTION

The current invention uses the principles of drug design to synthesize two new compounds CEE-1 and CEE-2 which have activities relevant to the treatment of asthma, allergy and inflammation. CEE-1 and CEE-2 are enhydrazone esters which uniquely combine three important attributes: ability to act directly and potently to inhibit human eosinophil degranulation, ability to inhibit directly and potently the degranulation of allergen-stimulated degranulation of mast cells and the ability to strongly and directly inhibit the release of tumor necrosis factor-alpha (TNF-α) in lipopolysacchrides (LPS)-stimulated macrophages.

These two novel compounds have a unique combination of properties that make them potentially useful in the treatment of asthma and other allergic diseases. They are also potentially useful in other non-allergic inflammatory diseases, such as rheumatoid arthritis.

### A. Synthesis of CEE-1 and CEE-2

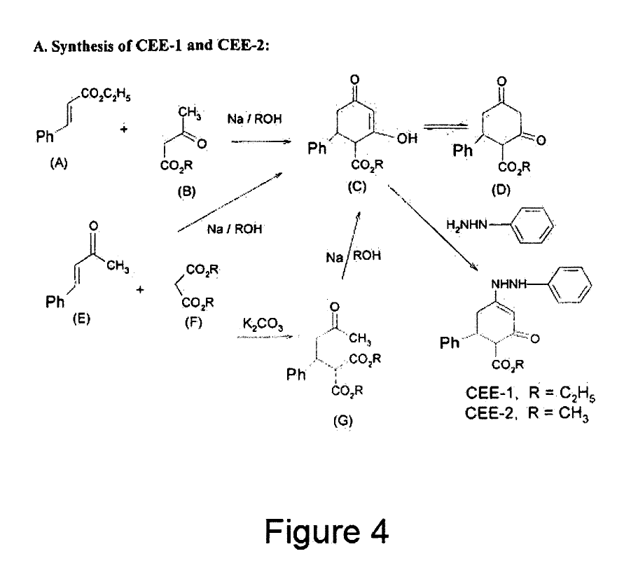

In FIG. 4, the cyclization reaction between trans-4-phenyI-3-buten-2-one (A) and alkyl acetoacetate (B) in the presence of freshly prepared sodium alkoxide gave the intermediate beta-hydroxy keto ester (C) which existed as two tautomers (C) and (D). Condensation of (C) with phenylhydrazine yielded the enhydrazone esters CEE-1 (R=Et) and CEE-2 (R=Me). Alternatively, benzylidene acetone (E) was reacted with dialkyl malonate (F) in freshly prepared sodium alkoxide to give the intermediate beta-hydroxy keto ester (C). The third route involved the reaction of (E) and (F) under mild conditions with potassium carbonate to give the adduct (G) which was cyclized in freshly prepared sodium alkoxide to give the intermediate beta-hydroxy keto ester (C). Thus, the synthesis of the intermediate beta-hydroxy keto ester (C) was unequivocal, and condensation with phenylhydrazine yielded CEE-1 (R=Et), and CEE-2 (R=Me).

The two compounds CEE-1 and CEE-2 are cyclohexenone derivatives which are stable solids at room temperature. Compound CEE-1 is ethyl 4-phenylhydrazinocyclohex-3-en-2-oxo-6-phenyl-1-oate with a molecular formula of C21H22O3N2 and molecular weight of 349.3. It has a melting point of 150-153° C., and it shows characteristic ultra violet absorption (intensity) in H2O: 294 nm (19183), 1M HCl: 283 nm (15993), and 1M NaOH: 319 nm (14548). Its infra red data (cm−1) are 1572, and 1666 for O═C—C═C—N system, 1730 for ester, and 3281 for NH.

Compound CEE-2 is methyl 4-phenylhydrazinocyclohex-3-en-2-oxo-6-phenyl-1-oate with a molecular formula of C20H20O3N2 and molecular weight of 336.39. It has a melting point of 179-182° C., and it shows characteristic ultra violet absorption (intensity) in H2O: 295 nm (24195), 1M HCl: 283 nm (19967), and 1M NaOH: 319 nm (16279). Its infra red data (cm−1) are 1492, 1569, for O═C—C═C—N system, 1733 for ester, 3266 for NH.

### B. Anti-Asthmatic and Anti-Allergic Effects of CEE-1 and CEE-2

Effect of Complement C5a-induced Degranulation of human blood eosinophils, and IgE-dependent degranulation of mast cells.

Human peripheral blood eosinophils were purified to >98% by the immunomagnetic method. Aliquots containing 2.5×104 cells were added to each well of a 96-well microplate. The cells were pre-incubated for 10 min with CEE-1 or CEE-2 or the drug solvent (DMSO) and subsequently stimulated with recombinant human complement C5a (30 nM) in the presence of cytochalasin B (5 μg/ml). The reaction was allowed to proceed for 30 min at 37° C. The amound of the granular eosinophil peroxidases (EPO) released into the supernatant (as index of degranulation) was determined by the o-phenylenediamine method. The amount of EPO released was expressed as a percentage of the total cell content.

The anti-allergic effect was tested on IgE-dependent mast cell degranulation using the rat basophilic leukemia (RBL-2H3) cell line model. The cells were passively sensitized with 0.5 μg/ml anti-DNP monoclonal IgE antibody for 1 h, washed twice to remove unbound antibody. They were then pre-treated with CEE-1 or CEE-2 or their solvent (DMSO) for 10 min before being stimulated with the specific antigen DNP-BSA (100 ng/ml). After 30 min incubation the amount of granular β-hexoseaminidase released into the supernatant (index of degranulation) was determined photometrically.

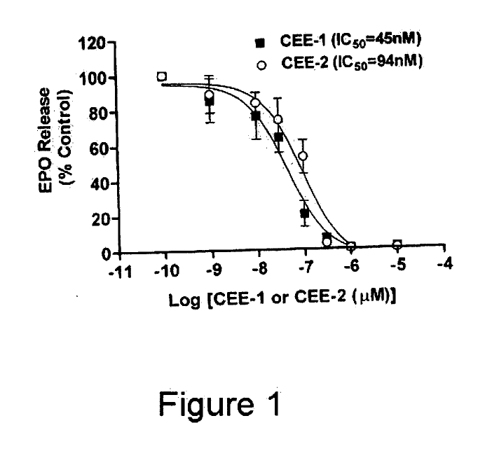

FIG. 1 displays the inhibition of complement C5a-stimulated degranulation (EPO release) of human eosinophils by CEE-1 and CEE-2. Cells were incubated with the drugs for 10 min before stimulation. Control un-inhibited releases were in the range 20-30% of total cell content. Results are mean+SD, n=4.

In untreated cells, C5a induced the release of 20-30% of the total cellular content of EPO. As shown in FIG. 1, pre-treatment of the cells with CEE-1 or CEE-2 resulted in a potent and highly effective inhibition of the EPO release. The concentration achieving 50% inhibition was 45 nM and 94 nM for CEE-1 and CEE-2, respectively and at the concentrations of 300 nM and above, both drugs completely abolished EPO release. Even at concentrations that abolished release, both compounds did not significantly affect the viability of the cells, thus riling out cellular toxicity as the cause of the inhibition.

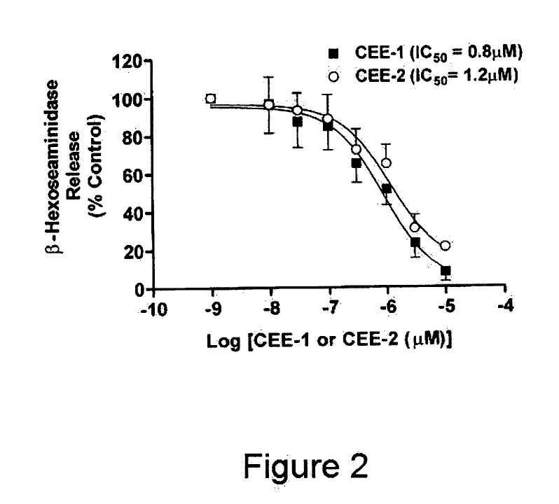

FIG. 2 displays the inhibition of antigen induced IgE-mediated degranulation (μ-hexoseaminidase release) of mast cells (RBL-2H3) by CEE-1 and CEE-2. Cells were incubated with the drugs for 10 min before stimulation. Control un-inhibited releases were in the range 15-26% of total cell content. Results are mean+SD, n=3.

As shown in FIG. 2, pre-treatment of passively-sensitized RBL-2H3 mast cells with CEE-1 and CEE-2 resulted in a strong concentration-dependent inhibition of β-hexoseaminidase release (index of degranulation). The concentrations achieving 50% inhibition were 0.8 μM and 1.2 μM for CEE-1 and CEE-2, respectively. At the highest concentrations, both drugs showed no cellular toxicity as determined by trypan blue exclusion test.

### C. Anti-Inflammatory Effect of EE-1 & CEE-2

The anti-inflammatory effect of CEE-1 and CEE-1 was tested on LPS-stimulated release of tumor necrotic factor-α (TNF-α) from mouse peritoneal macrophages. Thioglycollate-induced peritoneal macrophages were obtained by peritoneal lavage of Balb c mice. Macrophages purified by adherence to plastic plate were cultured in supplemented RPMI medium and pre-treated with CEE-1, CEE-2 or solvent for 30 min before being stimulated with LPS (1 μ*g/ml) for 18 h. The amount of TNF-α released into the supernatant was determined by ELISA using commercially available kits.

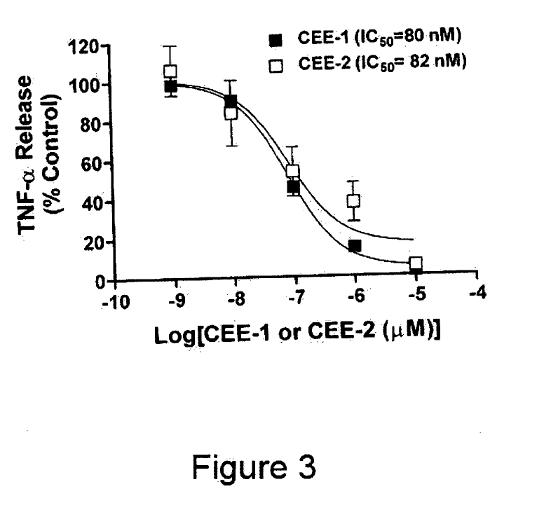

FIG. 3 displays the inhibitory effect of CEE-1 and CEE-2 on the release of TNF-α from LPS-stimulated mouse peritoneal macrophages. Cells were pre-treated with the drugs for 30 in before stimulation with LPS (1 μg/ml). The net un-inhibited releases were in the range 130-185 pg/ml. Values are means±SD, n=3.

As shown in FIG. 3, treatment of the cells with CEE-1 and CEE-2 strongly inhibited the release of TNF-α in a dose-dependent manner. The concentrations of the novel compounds that achieved 50% inhibition of the response were 80 nM and 82 nM, for CEE-1 and CEE-2, respectively. At 10 μM both compounds completely abolished the release. Overnight incubation of these cells with the two drugs showed no effect on their viability.

Many drugs have anti-asthma and anti-allergic effects, but these are either bronchodilators or inhibitors or release of allergic mediators or inflammatory mediators in general.

Due to the important role that eosinophils play in allergic diseases and asthma, drugs that have direct inhibitory effect on these cells are widely believed to hold a high potential for the effective treatment of these diseases. Previous studies have shown that no existing clinically used anti-allergic or anti-asthmatic drugs, at clinically-relevant concentrations, have any significant direct inhibitory effect on eosinophils degranulation (for example, Exeamuzie & Al-Hage 1998). To the best of our knowledge, this situation is still the same today.

Drugs that combine the ability to directly inhibit the degranulation of both mast cells and eosinophils at clinically-relevant concentrations are not currently available, and are highly sought after for the treatment of asthma and other allergic diseases.

### Advantages

The enhydrazone esters (CEE-1 and CEE-2) have a unique potent and direct inhibitory effect on the degranulation of human eosinophils The compounds combine the above effect with two other important activities: a direct inhibition of IgE-dependent mast cell degranulation and a direct inhibitory effect on cytokine release from macrophages. Thus, the drugs combine three potentially important activities—anti-asthmatic, anti-allergic and anti-inflammatory activities. The compounds have no direct toxic effect on these cells in vitro.

The methods of the present invention have been explained with reference to plurality of references the teachings of which are all incorporated herein by reference.

### Equivalents

From the foregoing description, one skilled in the art can easily ascertain the essential characteristics of this invention and, without departing from the spirit and scope thereof, can make various changes and modifications of the invention to adapt it to various usages and conditions. Such variations and changes may include, for example, altering the number of components in the housing or using equivalents. It is believed that such can be accomplished without excessive experimentation. In any case, any such variations are all claimed under the scope of this invention.

We claim:

1-8. (canceled)

9. A Method for the synthesis of enhydrazone esters of the general formula (I), wherein R is one or more of a set of ethyl (CEE-1) or methyl (CEE-2).

10. A compound or salt thereof represented by the general formula (I), where R is ethyl group (CEE-1).

11. A compound or salt thereof represented by the general formula (I), wherein R is methyl group (CEE-2).

12. The compound of claim 2 and its salts thereof as inhibitors of degranulation of both mast cells and eosinophils.

13. The compound of claim 2 and its salts thereof as inhibitors of cytokine release from monocytes/macrophages

14. The compound of claim 3 and its salts thereof as inhibitors of degranulation of both mast cells and eosinophils.

15. The compound of claim 3 and its salts thereof as inhibitors of cytokine release from monocytes/macrophages.

16. The compound of claim 2 or its salts, as a component of a composition for the prevention or treatment of asthma and allergic diseases, including but not limited to allergic rhinitis, allergic conjunctivitis, allergic eczema, atopic dermatitis, acute and chronic urticarias.

17. The compound of claim 2 or its salts, as a component of a composition for the prevention or treatment of inflammatory diseases, including, but not limited to rheumatoid arthritis, osteoarthritis, inflammatory bowel diseases, skin and ocular inflammation.

18. The compound of claim 3 or its salts, as a component of a composition for the prevention or treatment of asthma and allergic diseases, including but not limited to allergic rhinitis, allergic conjunctivitis, allergic eczema, atopic dermatitis, acute and chronic urticarias.

19. The compound of claim 3 or its salts, as a component of a composition for the prevention or treatment of inflammatory diseases, including, but not limited to rheumatoid arthritis, osteoarthritis, inflammatory bowel diseases, skin and ocular inflammation.
